SEQUENCE LISTING (3 sequences)

SEQ ID NO: 1 - protein, 20 aa, Artificial Sequence
  caveolin scaffolding domain peptide
DGIWKASFTTFTVTKYWFYR

SEQ ID NO: 2 - protein, 9 aa, Artificial Sequence
  amino acids 218-226 of alpha-PKC (alphaC2-4)
SLNPQWNET

SEQ ID NO: 3 - protein, 9 aa, Artificial Sequence
  myristoylated PKC-alpha/beta pseudosubstrate peptide
FARKGALRQ

(19) United States
(12) Patent Application Publication
(10) Pub. No.: US 2014/0213604 A1
(43) Pub. Date: Jul. 31, 2014

(54) PROTEIN KINASE C AS A TARGET FOR THE TREATMENT OF RESPIRATORY SYNCYTIAL VIRUS

(71) Applicants: SHYAM S. MOHAPATRA, LUTZ, FL (US); HOMERO GABRIEL VERGARA, TAMPA, FL (US)

(72) Inventors: SHYAM S. MOHAPATRA, LUTZ, FL (US); HOMERO GABRIEL VERGARA, TAMPA, FL (US)

(73) Assignee: UNIVERSITY OF SOUTH FLORIDA, TAMPA, FL (US)

(21) Appl. No.: 14/256,441

(22) Filed: Apr. 18, 2014

Related U.S. Application Data
(63) Continuation of application No. 12/384,384, filed on Apr. 3, 2009.
Application No. 12/384,384 is a division of application No. 12/079,595, filed on Mar. 26, 2008, now abandoned.
Application No. 12/079,595 is a division of application No. 10/734,548, filed on Dec. 12, 2003, now abandoned.
(60) Provisional application No. 60/319,780, filed on Dec. 13, 2002.

(51) Int. Cl. A61K 31/4741; A61K 45/06; A61K 31/265

(52) U.S. Cl. CPC A61K 31/4741; A61K 31/265; A61K 45/06
USPC 514/280; 514/510

(57) ABSTRACT

The subject invention concerns a method of inhibiting respiratory syncytial virus (RSV) infection in a patient by decreasing the endogenous protein kinase C (PKC) activity within the patient. Preferably, the preventative and therapeutic methods of the present invention involve administration of a PKC inhibitor. The present inventor has determined that decreasing normal endogenous PKC activity is inhibitory to RSV infection of human cells. The subject invention also pertains to pharmaceutical compositions containing a PKC inhibitor and a pharmaceutically acceptable carrier.

### CROSS-REFERENCE TO RELATED APPLICATIONS

The present application is a continuation of U.S. application Ser. No. 12/384,384, filed Apr. 3, 2009, which is a divisional of U.S. application Ser. No. 12/079,595, filed Mar. 26, 2008, now abandoned, which is a divisional of U.S. application Ser. No. 10/734,548, filed Dec. 12, 2003, now abandoned, which claims the benefit of U.S. Provisional Application Ser. No. 60/319,780, filed Dec. 13, 2002, each of which is hereby incorporated by reference herein in its entirety, including any figures, tables, or drawings.

### GOVERNMENT SUPPORT

This invention was made with government support under a research project supported by VA Merit Review Award. The government has certain rights in the invention.

### BACKGROUND OF INVENTION

Respiratory syncytial virus (RSV) is an important respiratory pathogen that produces an annual epidemic of respiratory illness primarily in infants, but also in adults, worldwide. RSV commonly causes bronchiolitis and exacerbates asthma, but it may also lead to severe life-threatening respiratory conditions resulting in prolonged hospitalization and death in high-risk individuals. The molecular pathology of RSV infection, specifically, the early events of virus-host interaction, is poorly understood.

RSV infection up-regulates the expression of several cytokines and chemokines, such as IL-1β, IL-6, IL-8, TNF-α, MIP1α, RANTES, and the adhesion molecule ICAM-1, in cultured epithelial cells, which are the main target of RSV infection in vivo. The elevated expression of these inflammatory molecules in RSV infection has been attributed to activation of the nuclear factor κB (NFκB). Additional transcription factors, such as C/EBP and AP1, MAPK regulate RSV-induced gene activation and have also been implicated; however, this has not been corroborated. Protein kinase C (PKC) consists of a family of serine/threonine kinases with at least 13 members. On the basis of their structures, the P1 family can be divided into three major subclasses: 1) the classical group A PKCs (cPKCs), comprising (alpha, beta I and II, and gamma (α, βI, βII, γ)) isozymes that are Ca⁺⁻ dependent and diacylglycerol (DAG sensitive, 2) the novel group B PKCs (nPKCs, comprising the delta, epsilon, nu, theta, and kappa isozymes that are Ca2⁺ independent and DAG sensitive, 3) the atypical group C isozymes comprising zeta, iota, and lambda (ξ, ι, λ) isozymes which are Ca2⁺ independent and DAG insensitive, and 4) the group D PKC μ isozyme that is similar to the group C isozymes but contains a specific signal peptide transmembrane domain. PKC contains two identifiable domains, a catalytic domain (the ATP binding site, blockable to staurosporir) and a regulatory domain (the phospholipid and diacylglycerol binding site, blockable by calphostin). Recently, several PKC isozymes expressed in the carcinoma cell line A549 were found activated in response to RSV infection, and PKC-α seems to participate in the activation of ERK-2. However, since a carcinoma cell line and non-purified RSV preparation were used in the aforementioned study, the PKC involvement in human primary epithelial cells remains unknown.

### BRIEF SUMMARY

The present invention provides materials and methods useful for inhibiting infections caused by respiratory syncytial virus (RSV). The subject invention concerns therapeutic methods for preventing or decreasing the severity of symptoms associated with an RSV infection by decreasing endogenous levels of protein kinase C (PKC) activity within the patient. Preferably, the endogenous levels of classical PKC isoform activity, such as PKC alpha activity, PKC beta activity, and/or PKC gamma activity are decreased within the patient. However, the endogenous levels of PKC epsilon activity, PKC zeta activity, and/or PKC theta activity can be decreased within the patient, either alternatively or in addition to, PKC alpha activity, PKC beta activity, and/or PKC gamma activity. The materials and methods of present invention are effective for treating or preventing RSV within a human or non-human animal.

In one aspect, the method of the present invention involves the administration of at least one PKC inhibitor to the patient. Preferably, the PKC inhibitor used in the methods, compositions, vectors, and host cells of the invention is an inhibitor of one or more classical PKC isoforms, such as an inhibitor of PKC alpha, PKC beta, and/or PKC gamma. Suitable PKC inhibitors include, but are not limited to, inhibitory chemical compounds, antisense oligonucleotide molecules, PKC pseudosubstrate peptides, and function-blocking antibodies or antibody fragments. The PKC inhibitor is preferably administered orally or intranasally to the epithelial mucosa of the respiratory system.

The present invention also pertains to pharmaceutical compositions comprising at least one PKC inhibitor, and a pharmaceutically acceptable carrier. The pharmaceutical compositions of the present invention are useful for preventing or decreasing the severity of symptoms associated with RSV infection. Preferably, the pharmaceutical composition of the present invention comprises at least one PKC inhibitor, at least one additional infection inhibiting agent, and a pharmaceutically acceptable carrier.

In one embodiment, the pharmaceutical composition comprises a vector containing a nucleotide sequence encoding a PKC inhibitor. Optionally, the vector can further include a promoter sequence operatively linked to the nucleotide sequence encoding the PKC inhibitor, permitting expression of the nucleotide sequence within a host cell. In another embodiment, the pharmaceutical composition comprises host cells that have been genetically modified with a nucleotide sequence encoding a PKC inhibitor such that the genetically modified cell produces the PKC inhibitor. In those pharmaceutical compositions of the present invention that comprise PKC inhibitors having a nucleic acid or amino acid component, the pharmaceutical compositions can include various agents that protect the nucleic acid or amino acid contents from degradation.

In another aspect, the present invention concerns vectors containing a nucleotide sequence encoding a PKC inhibitor. Optionally, the vector can further include a promoter sequence operatively linked to the nucleotide sequence encoding the PKC inhibitor, permitting expression of the nucleotide sequence within a host cell. In another aspect, the present invention includes host cells that have been genetically modified with a nucleotide sequence encoding a PKC inhibitor.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIGS. 1A and 1B show that AG 490 (a JAK2 inhibitor) and RO318220 (a PKC inhibitor) treatment substantially decrease RSV infection. NHBE cells were treated with different inhibitors for 2 hours at concentration as: AG 490 (50 μM), PD98059 (80 μM), RO318220 (3 μM), and Wortmannin (300 nM). DMSO was used as a mock control. The inhibitors were removed and the cells were infected with RSV for 2 hours. After the RSV was removed, the growth medium with the same concentration of inhibitors was added to the cells for 48 hours. The cells were rinsed with PBS prior to staining with FITC-labeled Anti-RSV monoclonal antibody (mab) (CHEMICON, Temecula, Calif.). Stained cells are shown in FIG. 1A. The total cells and RSV positive cells were counted randomly from 15 to 20 different spots. The results, shown in FIG. 1B, demonstrate that inhibitors reduced RSV infection 2- to 4-fold.

FIGS. 2A and 2B show that RSV causes changes in the relative amounts of PKC isoforms expressed in NHBE cells, and RSV infection of NHBE cells is inhibited by PKC inhibitors. NHBE cells were infected with purified RSV at an infectious dose of 1 MOI, and the infection was allowed to proceed for different time points (1 h, 2 h, and 8 h). Next, 20 μg of protein extracts from whole cell lysates were analyzed by Western-blot, and the PKC isoforms were probed using specific mouse mab. Results of the Western-blot are shown in FIG. 2A. Confluent NHBE cells were treated with PKC inhibitors at different doses for 30 minutes before infecting them with RSV at an infectious dose of 1 MOI. The infection was allowed to proceed for 16 hours and infected cells were detected by single cell immunofluorescence assays. The percentage of inhibition was calculated with respect to control (DMSO). The percentage of infected cells is shown in FIG. 2B. The values are means±S.D. of three different experiments.

FIGS. 3A-3F show that PKC-α/β pseudosubstrate peptide did not interfere with the RSV binding to NHBE cells. Confluent NHBE cells were treated with no peptide (FIG. 3A), control peptide (FIGS. 3B and 3C), or inhibitor peptide (FIGS. 3D, 3E, and 3F) at the indicated concentrations for 30 minutes before being infected with RSV at an infectious dose of 1 MOI. The infection was allowed to proceed for 24 hours. Next, cell culture monolayers were detached by trypsin treatment, and single cells suspensions were processed for FACS. The RSV-infected cells were detected by a FITC-labeled mouse monoclonal anti-RSV N protein Ab.

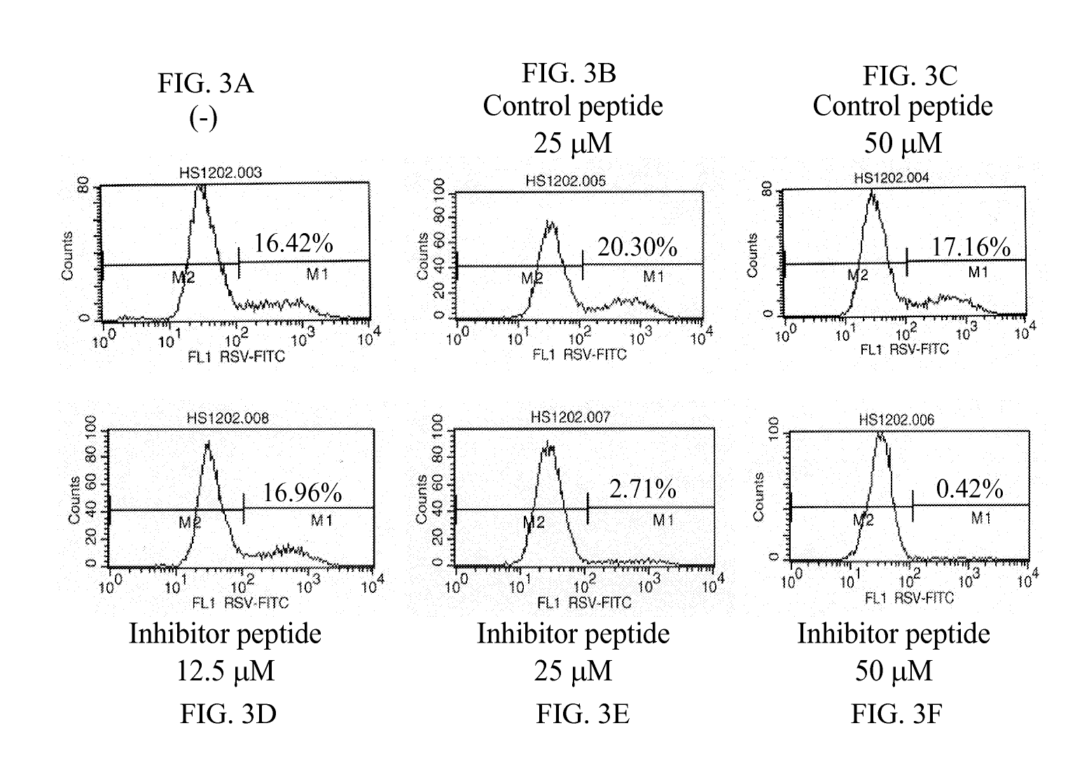

FIGS. 4A-4F show that PKC-α co-localizes with RSV at early stages of infection. Confluent NHBE cells grown on 8-well chambered slides were exposed to RSV at an infectious dose of 20 MOI for 10 min before processing them for immunocytofluorescence. NHBE cells were fixed with 4% paraformaldehyde and then stained with mouse monoclonal anti-PKC-α antibody (green), goat polyclonal anti-RSV antibody (red), and DAPI (blue, nucleus staining) Fluorescence images were taken by cooled camera device (CCD) under respective dual filter mode (either green/blue or red/blue) and triple filter mode (merge). FIGS. 4A-4C show non-infected cells. FIGS. 4D-4F show RSV-infected cells.

FIG. 5 shows an increase of Phospho-PKC-α and its association with RSV particles contacting NHBE cells. Confluent NHBE cells grown on 8-well chambered slides were exposed to RSV at an infectious dose of 20 MOI for 10 min. As negative controls, cells were either pre-treated with PKC-α/β pseudosubstrate inhibitor at 50 μM for 30 min before infection or exposed to sham treatment (CENTRICON's filtrate obtained from purified-RSV). NHBE cells were fixed with 4% paraformaldehyde and then stained with mouse monoclonal anti-PKC-α antibody (green), goat polyclonal anti-RSV antibody (red), and DAPI (blue, nucleus staining) Confocal images were taken using laser excitation sources for Alexa-488 (green) or Alexa-555 (red) and assembled using ADOBE PHOTOSHOP version software 7.01.

FIGS. 6A-6D show that of PKC-α activity blocks viral fusion. Confluent NHBE cells seeded on 8-well chambered slides were pre-incubated with PKC-α/β pseudosubstrate peptide for 30 minutes at the indicated concentrations before exposing the cells to Octadecyl rhodamine B (R18)-labeled RSV (5000 RSV particles/cell). The infection was allowed to proceed for 30 minutes at 37° C. After removal of the unattached virus, cells were imaged using a fluorescence microscope.

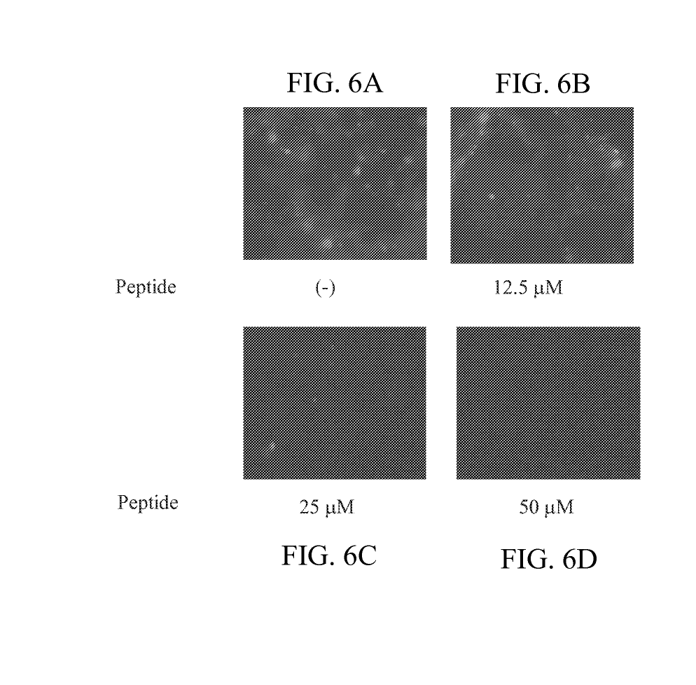

FIGS. 7A-7C show that treatment of NHBE cells with PKC-α/β pseudosubstrate peptide alters the RhoA appropriate location for successful RSV infection. Confluent NHBE cells seeded on 8-well chambered slides were treated with either PKC-α/β pseudosubstrate peptide or vehicle (HEPES saline buffer) for 30 minutes at 50 μM before exposing the cells to RSV at an infectious dose of 20 MOI for 10 min. Large arrows indicate RhoA present at membrane after RSV infection. Arrow heads indicate restricted location of RhoA induced by PKC-α/β pseudosubstrate peptide.

### BRIEF DESCRIPTION OF THE SEQUENCES

SEQ ID NO:1 is a caveolin scaffolding domain peptide.
SEQ ID NO:2 is amino acids 218-226 of αPKC (αC2-4).
SEQ ID NO:3 is a myristoylated PKC-α/β pseudosubstrate peptide.

### DETAILED DISCLOSURE

The subject invention concerns a method of inhibiting a respiratory syncytial virus (RSV) infection within a patient by decreasing the endogenous levels of PKC activity within the patient. Preferably, the endogenous levels of classical PKC isoform activity are decreased, such as PKC alpha activity, PKC beta activity, and/or PKC gamma activity. More, preferably, the endogenous levels of PKC alpha isozyme activity are decreased within the patient. However, the endogenous levels of other PKC isoforms can be decreased within the patient, either alternatively or in addition to the activities of one or more of the classical PKC isoforms.

In preferred embodiments, the activity of one or more PKC isoforms that are found at membrane structures known as caveolae (Anderson, R. G. W., Ann. Rev. Biochem., 67:199-225, 1998), and/or that contribute to caveolae formation, is decreased. For example, selective or non-selective inhibitors of such isoforms can be administered to the patient or used in the compositions, vectors, and host cells of the invention.

In another aspect, the present invention concerns a pharmaceutical composition comprising at least one PKC inhibitor and a pharmaceutically acceptable carrier. Preferably, the pharmaceutical composition comprises at least one additional infection inhibiting agent. Preferably, the additional infection inhibiting agent is an antiviral agent, such as an RSV inhibiting agent.

The methods, compositions, vectors, and host cells of the present invention can employ any PKC inhibitor, including non-isozyme-specific PKC inhibitors and isozyme-specific PKC inhibitors. Preferably, the inhibitor selectively inhibits one or more classical type PKC present in the patient (i.e., does not inhibit other PKC non-classical isoforms). A wide variety of suitable inhibitors may be employed, guided by art-recognized criteria such as efficacy, toxicity, stability, specificity, half-life, etc. Information about PKC inhibitors, and methods for their preparation are readily available in the art. For example, different kinds of PKC inhibitors and their preparation are described in U.S. Pat. Nos. 5,621,101; 5,621,098; 5,616,577; 5,578,590; 5,545,636; 5,491,242; 5,488,167; 5,481,003; 5,461,146; 5,270,310; 5,216,014; 5,204,370; 5,141,957; 4,990,519; and 4,937,232. Preferably, the PKC inhibitor used in the methods, compositions, vectors, and host cells of the present invention effectively inhibit the alpha isozyme.

In general, PKCs contain a regulatory and a catalytic domain. In addition to targeting the catalytic domain for inhibition by using, for example, pseudosubstrate peptides or chemical compounds which block the ATP-binding site, any means of interfering with PKC translocation to places where these enzymes are required for accomplishing their function are also a target for inhibition. Moreover, differential localization of individual isozymes, namely activation-induced binding of PKC to anchoring proteins, provides the capability of using isozyme-specific PKC inhibitors that are more likely to overcome the toxicity encountered with the first generation inhibitors that target conserved sites within the regulatory and catalytic domains. For example, peptides obtained from the sequence of RACK (Receptors for Activated C-Kinase) specifically inhibit both PKC binding to RACK and, consequently, its activation.

In particular embodiments, the PKC inhibitor is elected from competitive inhibitors for the PKC ATP-binding site, including staurosporine and its bisindolylmaleimide derivatives, Ro-31-7549, Ro-31-8220, Ro-31-8425, Ro-32-0432 (bisindolylmaleimide tertiary amine), and Sangivamycin (Tamaoki, T. et al., Biochem. Biophys. Res. Commun. 135:397-402, 1986; Meyer, T. et al., Int. J. Cancer 43:851-856, 1989); drugs which interact with the PKC's regulatory domain by competing at the binding sites of diacylglycerol and phorbol esters, such as calphostin C (Kobayashi, E. et al., Biochem. Biophys. Res. Commun. 159:548-553, 1989), safingol (L-threo-dihydrosphingosine), D-erythro-sphingosine; drugs which target the catalytic domain of PKC such as chelerythrine chloride, and Melittin; drugs which inhibit PKC by covalently binding to PKC upon exposure to UV lights, such as dequalinium chloride; drugs which specifically inhibit Ca-dependent PKC such as Go6976, Go6983, Go7874 and other homologs, polymyxin B sulfate; drugs comprising competitive peptides derived from PKC sequence; and other PKC inhibitors such as cardiotoxins, ellagic acid, HBDDE, 1-O-Hexadecyl-2-O-methyl-rac-glycerol, Hypercin, K-252, NGIC-J, phloretin, piceatannol, tamoxifen citrate, flavopiridol (L86-8275), and bryostatin 1 (Macrocyclic lactone). Other minoacridines (Hannun, Y. A. and R. M. Bell, J. Biol. Chem. 263:5124-5131, 1988), sphingolipids (Hannun, Y. A. et al., J. Biol. Chem. 264:9960-9966, 1989), bisindolylmaleimides (Toullec, D. et al., J. Biol. Chem. 266:15771-15781, 1991), and isoquinolinesulfonamides (Hidaka, H. et al., Biochemistry 23:5036-5041, 1984) have also been identified as PKC inhibitors. In addition, PKC antisense or plasmids encoding siRNA that targets PKC, which can be complexed with nanoparticles specifically addressed to bronchial epithelium (a primary target for RSV infection), can also be used. It is also possible to use plasmids encoding the regulatory domain of PKC, such as PKC-alpha, which is a very specific inhibitor for PKC translocation and activation.

Additional inhibitors of PKC can be identified using assays that measure the activation, intracellular translocation, binding to intracellular receptors (e.g., RACKs) or catalytic activity of PKC. Traditionally, the kinase activity of PKC family members has been assayed using at least partially purified PKC in a reconstituted phospholipid environment with radioactive ATP as the phosphate donor and a histone protein or a short peptide as the substrate (Kitano, T. et al., Meth. Enzymol. 124, 349-352, 1986; Messing, R. O. et al., J. Biol. Chem. 266, 23428-23432, 1991). More recent improvements include a rapid, highly sensitive chemiluminescent assay that measures protein kinase activity at physiological concentrations and can be automated and/or used in high-throughput screening (Lehel, C. et al., Anal. Biochem. 244, 340-346, 1997) and an assay using PKC in isolated membranes and a selective peptide substrate that is derived from the MARCKS protein (Chakravarthy, B. R. et al., Anal. Biochem. 196, 144-150, 1991). Inhibitors that affect the intracellular translocation of PKC can be identified by assays in which the intracellular localization of PKC is determined by fractionation (Messing, R. O. et al., Biol. Chem. 266, 23428-23432, 1991) or immunohistochemistry (U.S. Pat. No. 5,783,405). To identify an inhibitor of PKC alpha, for example, the assays are performed with PKC alpha as the target. The selectivity of such PKC alpha inhibitors can be determined by comparing the effect of the inhibitor on PKC alpha with its effect on other PKC isozymes.

In another aspect, the subject invention concerns a method of treating or preventing an RSV infection within a patient by decreasing the in vivo concentration of PKC within the patient, thereby inhibiting the RSV infection. Thus, in one aspect, the methods and compositions of the present invention are directed to decreasing the in vivo concentration of PKC. Preferably, the in vivo concentration of PKC polypeptide is decreased by interfering with or down-regulating the functional expression of the nucleotide sequence encoding PKC, as gene therapy.

The in vivo concentration of PKC can be decreased, for example, by exogenous administration of an agent, such as an antisense oligonucleotide molecule, that interferes with expression of PKC. For example, oligonucleotides can be designed to hybridize to PKC mRNA, such as human PKC mRNA, thereby interfering with translation. The interfering oligonucleotide can be administered to a patient's cells in vivo or in vitro (including ex vivo, genetically modifying the patient's own cells ex vivo and subsequently administering the modified cells back into the patient). Stable transfection of antisense PKC alpha cDNA has been carried out in cytomegalovirus promoter-based expression vectors to specifically decrease expression of PKC-alpha protein (Godson et al. J. Biol. Chem. 268:11946-11950, 1993) disclosed use of. Transfection of the human glioblastoma cell line, U-87, has been achieved with vectors expressing RNA antisense to PKC alpha inhibits growth of the glioblastoma cells in vitro and in vivo (Ahmad et al., Neurosurg. 35:904-908, 1994). A peptide corresponding to the pseudo-substrate region of PKC zeta and oligonucleotides antisense to this isozyme are known (International PCT Application WO 93/20101). A mutant form of PKC associated with tumors has been identified and oligonucleotide sequences complementary to the mutant form have been developed (International PCT Application WO 94/29455). Methods of modulating PKC expression using oligonucleotides targeted to PKC are also disclosed in U.S. patent publication 2003/0148989 (Bennet F. C. et al.).

In the present invention, the oligonucleotide is designed to bind directly to mRNA or to a gene, ultimately modulating the amount of PKC protein made from the gene. This relationship between an oligonucleotide and its complementary nucleic acid target to which it hybridizes is commonly referred to as “antisense”. “Targeting” an oligonucleotide to a chosen nucleic acid target, in the context of this invention, is a multi-step process. The process usually begins with identifying a nucleic acid sequence whose function is to be modulated. This may be, as examples, a cellular gene (or mRNA made from the gene) whose expression is associated with a particular disease state, or a foreign nucleic acid from an infectious agent. In the present invention, the target is a nucleic acid encoding PKC; in other words, a PKC gene or mRNA expressed from a PKC gene. The targeting process also includes determination of a site or sites within the nucleic acid sequence for the oligonucleotide interaction to occur such that the desired effect--modulation of gene expression--will result. Once the target site or sites have been identified, oligonucleotides are chosen which are sufficiently complementary to the target, i.e., hybridize sufficiently well and with sufficient specificity, to give the desired modulation.

Inhibition of PKC expression can be measured in ways which are routine in the art, for example by Northern blot assay of mRNA expression or Western blot assay of protein expression as taught in the examples of the instant application. Effects on cell proliferation or tumor cell growth can also be measured, as taught in the examples of the instant application.

“Hybridization”, in the context of the present invention, means hydrogen bonding, also known as Watson-Crick base pairing, between complementary bases, usually on opposite nucleic acid strands or two regions of a nucleic acid strand. Guanine and cytosine are examples of complementary bases which are known to form three hydrogen bonds between them. Adenine and thymine are examples of complementary bases which form two hydrogen bonds between them. “Specifically hybridizable” and “complementary” are terms which are used to indicate a sufficient degree of complementarity such that stable and specific binding occurs between the DNA or RNA target and the oligonucleotide.

It is understood that an oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

In the context of the present invention, the term “oligonucleotide” refers to a polynucleotide formed from naturally occurring nucleobases and pentofuranosyl (sugar) groups joined by native phosphodiester bonds. This term effectively refers to naturally occurring species or synthetic species formed from naturally occurring subunits or their close homologs. The term “oligonucleotide” may also refer to moieties which function similarly to naturally occurring oligonucleotides but which have non-naturally occurring portions. Thus, oligonucleotides may have altered sugar moieties, nucleobases or inter-sugar (“backbone”) linkages. Such modified or substituted oligonucleotides are often preferred over native forms because of properties such as, for example, enhanced cellular uptake, enhanced target binding affinity and increased stability in the presence of nucleases.

Specific examples of some preferred oligonucleotides envisioned for this invention are those which contain intersugar backbone linkages such as phosphotriesters, methyl phosphonates, short chain alkyl or cycloalkyl intersugar linkages or short chain heteroatomic or heterocyclic intersugar linkages. Most preferred are phosphorothioates and those with CH₂—NH—O—CH₂, CH₂—N(CH₃)—O—CH₂ (known as the methylene(methylimino) or MMI backbone), CH₂—O—N(CH₃)—CH₂, CH₂—N(CH₃)—N(CH₃)—CH₂ and O—N(CH₃)—CH₂—CH₂ backbones (where phosphodiester is O—P—O—CH₂). Phosphorothioates are also most preferred. Also preferred are oligonucleotides having morpholino backbone structures. Summerton, J. E. and Weller, D. D., U.S. Pat. No. 5,034,506. In other preferred embodiments, such as the peptide nucleic acid (PNA—referred to by some as “protein nucleic acid”) backbone, the phosphodiester backbone of the oligonucleotide may be replaced with a polyamide backbone wherein nucleosidic bases are bound directly or indirectly to aza nitrogen atoms or methylene groups in the polyamide backbone (see, e.g., Nielsen, P. E. et al. Science 254:1497, 1991). In accordance with other preferred embodiments, the phosphodiester bonds are substituted with structures that are chiral and enantiomerically specific. Persons of ordinary skill in the art will be able to select other linkages for use in practice of the invention.

Oligonucleotides inhibiting PKC expression may also include species having at least one modified nucleotide base. Thus, purines and pyrimidines other than those normally found in nature may be so employed. Similarly, modifications on the pentofuranosyl portion of the nucleotide subunits may also be effected, as long as the essential tenets of this invention are adhered to. Examples of such modifications are 2′-O-alkyl- and 2′-halogen-substituted nucleotides. Some specific examples of modifications at the 2′ position of sugar moieties which are useful in the present invention are OH, SH, SCH₃, F, OCN, O(CH₂)ₙNH₂ or O(CH₂)ₙCH₃ where n is from 1 to about 10; C₁ to C₁₀ lower alkyl, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. One or more pentofuranosyl groups may be replaced by another sugar, by a sugar mimic such as cyclobutyl or by another moiety which takes the place of the sugar.

Chimeric or “gapped” oligonucleotides inhibiting PKC expression may also be used. These oligonucleotides contain two or more chemically distinct regions, each comprising at least one nucleotide. Typically, one or more region comprises modified nucleotides that confer one or more beneficial properties, for example, increased nuclease resistance, increased uptake into cells or increased binding affinity for the RNA target. One or more unmodified or differently modified regions retain the ability to direct RNase H cleavage.

The oligonucleotides in accordance with the present invention preferably comprise from about 5 to about 50 nucleotides, although larger oligonucleotides may be used. As will be appreciated by those skilled in the art, a nucleotide is a base-sugar combination (or a combination of analogous structures) suitably bound to an adjacent nucleotide unit through phosphodiester or other bonds forming a backbone structure.

The oligonucleotides used in accordance with this invention may be conveniently and routinely made through the well-known technique of solid phase synthesis. Equipment for such synthesis is sold by several vendors including APPLIED BIOSYSTEMS. Any other means for such synthesis may also be employed; the actual synthesis of the oligonucleotides is well within the talents of those skilled in the art. It is also well known to use similar techniques to prepare other oligonucleotides, such as phosphorothioates or alkylated derivatives. It is also well known to use similar techniques and commercially available modified amidites and controlled-pore glass (CPG) products such as biotin, fluorescein, acridine or psoralen-modified amidites and/or CPG (available from GLEN RESEARCH, Sterling, Va.) to synthesize fluorescently labeled, biotinylated or other modified oligonucleotides such as cholesterol-modified oligonucleotides. Other modified and substituted oligomers can be similarly synthesized.

In accordance with this invention, persons of ordinary skill in the art will understand that messenger RNA includes not only the information to encode a protein using the three letter genetic code, but also associated ribonucleotides which form a region known to such persons as the 5′-untranslated region, the 3′-untranslated region, the 5′ cap region and intron/exon junction ribonucleotides. Thus, oligonucleotides may be formulated in accordance with the present invention which are targeted wholly or in part to these associated ribonucleotides as well as to the informational ribonucleotides. In preferred embodiments, the oligonucleotide is specifically hybridizable with a transcription initiation site, a translation initiation site, a 5′ cap region, an intron/exon junction, coding sequences or sequences in the 5′- or 3′-untranslated region.

The oligonucleotides used in the methods and compositions of the present invention are designed to be hybridizable with messenger RNA derived from the PKC gene. Such hybridization, when accomplished, interferes with the normal roles of the messenger RNA to cause a modulation of its function in the cell. The functions of messenger RNA to be interfered with may include all vital functions such as translocation of the RNA to the site for protein translation, actual translation of protein from the RNA, splicing of the RNA to yield one or more mRNA species, and possibly even independent catalytic activity which may be engaged in by the RNA. The overall effect of such interference with the RNA function is to modulate expression of the PKC gene. The PKC inhibitor used in accordance with this invention can also be an antibody that is specifically reactive with PKC, and which inhibits the function of PKC. The PKC inhibitor can be an antibody or a fragment thereof, e.g., an antigen binding portion thereof, that inhibits the function of one or more PKC isoforms, such as PKC alpha. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. Sequences of Proteins of Immunological Interest, Fifth Edition, U.S. Department of Health and Human Services, NIH Publication No. 91-3242, 1991; and Chothia, C. et al. J. Mol. Biol. 196:901-917, 1987). Examples of antibodies that are specifically reactive with PKC are disclosed in published U.S. patent application 2002/0165158 (King).

The antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

The term “antigen-binding fragment” of an antibody (or simply “antibody portion,” or “fragment”), as used herein, refers to one or more fragments of a full-length antibody that retain the ability to specifically bind to an antigen. Examples of binding fragments encompassed within the term “antigen-binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature 341:544-546, 1989), which consists of a VH domain; and (vi) an isolated complementarity determining region (CDR). Furthermore, although the two domains of the Fv fragment, VL and VH, are coded for by separate nucleic acids, they can be joined, using recombinant methods, by a synthetic linker that enables them to be made as a single protein chain in which the VL and VH regions pair to form monovalent molecules (known as single chain Fv (scFv); see e.g., Bird et al., Science 242:423-426, 1988; and Huston et al., Proc. Natl. Acad. Sci. USA 85:5879-5883, 1988). Such single chain antibodies are also intended to be encompassed within the term “antigen-binding fragment” or “fragment” of an antibody. These antibody fragments are obtained using conventional techniques known to those with skill in the art, and the fragments are screened for utility in the same manner as are intact antibodies. The term “monoclonal antibody” or “monoclonal antibody composition”, as used herein, refers to a population of antibody molecules that contain only one species of an antigen binding site capable of immunoreacting with a particular epitope. A monoclonal antibody composition thus typically displays a single binding affinity for a particular protein with which it immunoreacts.

Anti-protein/anti-peptide antisera or monoclonal antibodies can be made as described herein by using standard protocols (See, for example, Antibodies: A Laboratory Manual ed. by Harlow and Lane (Cold Spring Harbor Press: 1988)).

PKC, such as PKC alpha, or a portion or fragment thereof, can be used as an immunogen to generate antibodies that bind the component using standard techniques for polyclonal and monoclonal antibody preparation. The full-length component protein can be used or, alternatively, antigenic peptide fragments of the component can be used as immunogens.

Typically, a peptide is used to prepare antibodies by immunizing a suitable subject, (e.g., rabbit, goat, mouse or other mammal) with the immunogen. An appropriate immunogenic preparation can contain, for example, a recombinant PKC, e.g., PKC alpha, or a chemically synthesized PKC. The nucleotide and amino acid sequences of PKC, e.g., PKC alpha, are known. The preparation can further include an adjuvant, such as Freund's complete or incomplete adjuvant, or similar immunostimulatory agent. Immunization of a suitable subject with an immunogenic component or fragment preparation induces a polyclonal antibody response.

Additionally, antibodies produced by genetic engineering methods, such as chimeric and humanized monoclonal antibodies, comprising both human and non-human portions, which can be made using standard recombinant DNA techniques, can be used. Such chimeric and humanized monoclonal antibodies can be produced by genetic engineering using standard DNA techniques known in the art, for example using methods described in U.S. Pat. No. 4,816,567; Better et al., Science 240:1041-1043, 1988; Liu et al., PNAS 84:3439-3443, 1987; Liu et al., J. Immunol. 139:3521-3526, 1987; Sun et al. PNAS 84:214-218, 1987; Nishimura et al., Canc. Res. 47:999-1005, 1987; Wood et al., Nature 314:446-449, 1985; and Shaw et al., J. Natl. Cancer Inst. 80:1553-1559, 1988); Morrison, S. L., Science 229:1202-1207, 1985; Oi et al., BioTechniques 4:214, 1986; U.S. Pat. No. 5,225,539; Jones et al., Nature 321:552-525, 1986; Verhoeyan et al., Science 239:1534, 1988; and Beidler et al., J. Immunol. 141:4053-4060, 1988.

In addition, a human monoclonal antibody directed against PKC, e.g., PKC alpha, can be made using standard techniques. For example, human monoclonal antibodies can be generated in transgenic mice or in immune deficient mice engrafted with antibody-producing human cells. Methods of generating such mice are described, for example, in Wood et al. PCT publication WO 91/00906, Kucherlapati et al. PCT publication WO 91/10741; Lonberg et al. PCT publication WO 92/03918; Kay et al. PCT publication WO 92/03917; Kay et al. PCT publication WO 93/12227; Kay et al. PCT publication 94/25585; Rajewsky et al. PCT publication WO 94/04667; Ditullio et al. PCT publication WO 95/17085; Lonberg, N. et al. Nature 368:856-859, 1994; Green, L. L. et al. Nature Genet. 7:13-21, 1994; Morrison, S. L. et al. Proc. Natl. Acad. Sci. USA 81:6851-6855, 1994; Bruggeman et al. Year Immunol. 7:33-40, 1993; Choi et al. Nature Genet. 4:117-123, 1993; Tuaillon et al. PNAS 90:3720-3724, 1993; Bruggeman et al. (1991) Eur. J. Immunol. 21:1323-1326, 1991; Duchosal et al. PCT publication WO 93/05796; U.S. Pat. No. 5,411,749; McCune et al. Science 241:1632-1639, 1988, Kamel-Reid et al. Science 242:1706, 1988; Spanopoulou Genes & Development 8:1030-1042, 1994; Shinkai et al. Cell 68:855-868, 1992. A human antibody-transgenic mouse or an immune deficient mouse engrafted with human antibody-producing cells or tissue can be immunized with PKC, e.g., PKC alpha, or an antigenic peptide thereof, and splenocytes from these immunized mice can then be used to create hybridomas. Methods of hybridoma production are well known.

Human monoclonal antibodies can also be prepared by constructing a combinatorial immunoglobulin library, such as a Fab phage display library or a scFv phage display library, using immunoglobulin light chain and heavy chain cDNAs prepared from mRNA derived from lymphocytes of a subject (see, e.g., McCafferty et al. PCT publication WO 92/01047; Marks et al. J. Mol. Biol. 222:581-597, 1991; and Griffiths et al. EMBO J 12:725-734, 1993). In addition, a combinatorial library of antibody variable regions can be generated by mutating a known human antibody. For example, a variable region of a human antibody known to bind a PKC, e.g., PKC alpha, can be mutated by, for example, using randomly altered mutagenized oligonucleotides, to generate a library of mutated variable regions which can then be screened to bind to PKC, e.g., PKC alpha. Methods of inducing random mutagenesis within the CDR regions of immunoglobin heavy and/or light chains, methods of crossing randomized heavy and light chains to form pairings and screening methods can be found in, for example, Barbas et al. PCT publication WO 96/07754; Barbas et al. Proc. Nat'l Acad. Sci. USA 89:4457-4461, 1992.

The PKC inhibitor used in the methods, composition vectors, and host cells of the present invention can also be a polypeptide exhibiting PKC inhibitory activity, such as a PKC pseudosubstrate peptide. An example of a PKC pseudosubstrate sequence that inhibits RSV infection in a dose-responsive manner is described in the Examples section. The activity of PKC, such as PKC alpha, can be specifically inhibited using other peptides as well, such as αC2-4 (amino acids 218-226 of αPKC (SLNPQWNET) (SEQ ID NO: 2) (Souroujon and Mochly-Rosen, Nat. Biotechnol. 1998; 16: 919-924; Disatnik, M H, J Cell Sci. 2002 May 15;115(Pt 10):2151-63); Methods Enzymol. 2002;345:470-89). Various means for delivering polypeptides to a cell can be utilized to carry out the methods of the subject invention. For example, protein transduction domains (PTDs) can be fused to the polypeptide, producing a fusion polypeptide, in which the PTDs are capable of transducing the polypeptide cargo across the plasma membrane (Wadia, J. S. and Dowdy, S. F., Curr. Opin. Biotechnol., 2002, 13(1)52-56). Examples of PTDs include the Drosophila homeotic transcription protein antennapedia (Antp), the herpes simples virus structural protein VP22, and the human immuno-deficiency virus 1 (HIV-1) transcriptional activator Tat protein.

According to the method of RSV inhibition of the subject invention, recombinant cells can be administered to a patient, wherein the recombinant cells have been genetically modified to express a nucleotide sequence encoding a PKC inhibitory polypeptide. If the cells to be genetically modified already express a nucleotide sequence encoding a PKC inhibitor polypeptide, the genetic modification can serve to enhance or increase expression of the nucleotide sequence beyond the normal or constitutive amount (e.g., “overexpression”).

The method of RSV inhibition of the subject invention can be used to treat a patient suffering from an RNA virus infection, or as a preventative of RSV infection (i.e., prophylactic treatment). As used herein, the terms “treat” or “treatment” are intended to include prevention of RSV infection, as well as inhibition of an existing RSV infection. According to the methods of the subject invention, various other compounds, such as other antiviral agents, can be administered in conjunction with (before, during, or after) decreasing the in vivo PKC activity within the patient. Thus, various compositions and methods for preventing or treating RSV infection can be used in conjunction with the compositions and methods of the subject invention, such as those described in U.S. Pat. No. 6,489,306, filed Feb. 23, 1999, and U.S. published patent application Serial No. 2003/00068333, filed Feb. 12, 2002, which are incorporated herein by reference in their entirety. For example, nucleotide sequences encoding a PKC inhibitory polypeptide can be conjugated with chitosan, a biodegradable, human-friendly cationic polymer that increases mucosal absorption of the composition without any adverse effects, as described in published U.S. patent application no. 2003/00068333.

The polynucleotide can be formulated in the form of nanospheres with chitosan. Chitosan allows increased bioavailability of the DNA because of protection from degradation by serum nucleases in the matrix and thus has great potential as a mucosal gene delivery system, for example. Chitosan exhibits various beneficial effects, such as anticoagulant activity, wound-healing properties, and immunostimulatory activity, and is capable of modulating immunity of the mucosa and bronchus-associated lymphoid tissue.

Nucleotide, polynucleotide, or nucleic acid sequences(s) are understood to mean, according to the present invention, either a double-stranded DNA, a single-stranded DNA, products of transcription of the said DNAs (e.g., RNA molecules), or corresponding RNA molecules that are not products of transcription. The nucleic acid sequences, polynucleotides, or nucleotide sequences used in the invention can be isolated, purified (or partially purified), by separation methods including, but not limited to, ion-exchange chromatography, molecular size exclusion chromatography, affinity chromatography, or by genetic engineering methods such as amplification, cloning or subcloning.

Optionally, the polynucleotide encoding the PKC inhibitory polypeptides can also contain one or more polynucleotides encoding heterologous polypeptides (e.g., tags that facilitate purification of the polypeptides of the invention (see, for example, U.S. Pat. No. 6,342,362, hereby incorporated by reference in its entirety; Altendorf et al. J. of Experimental Biology 203:19-28, 1999-WWW, 2000; Baneyx Biotechnology 10:411-21, 1999; Eihauer et al. J. Biochem Biophys Methods 49:455-65, 2001; Jones et al. J. of Chromatography A. 707:3-22, 1995; Margolin Methods 20:62-72, 2000; Puig et al. Methods 24:218-29, 2001; Sassenfeld TibTech 8:88-93, 1990; Sheibani Prep. Biochem. & Biotechnol. 29(1):77-90, 1999; Skerra et al. Biomolecular Engineering 16:79-86, 1999; Smith The Scientist 12(22):20, 1998; Smyth et al. Methods in Molecular Biology, 139:49-57, 2000; Unger The Scientist 11(17):20, 1997, each of which is hereby incorporated by reference in their entireties), or commercially available tags from vendors such as such as STRATAGENE (La Jolla, Calif.), NOVAGEN (Madison, Wis.), QIAGEN, Inc., (Valencia, Calif.), or INVITROGEN (San Diego, Calif.).

Other aspects of the invention provide vectors containing one or more of the polynucleotides encoding PKC inhibitory polypeptides. The vectors can be vaccine, replication, or amplification vectors. In some embodiments of this aspect of the invention, the polynucleotides are operably associated with regulatory elements capable of causing the expression of the polynucleotide sequences. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, lentiviruses, fowl pox viruses, pseudorabies viruses and retroviruses, and vectors derived from combinations of the aforementioned vector sources, such as those derived from plasmid and bacteriophage genetic elements (e.g., cosmids and phagemids). Preferably, the vector is an adenoaviral vector or adeno-associated virus vector.

As indicated above, vectors of this invention can also comprise elements necessary to provide for the expression and/or the secretion of the PKC inhibitor encoded by the nucleotide sequences in a given host cell. The vector can contain one or more elements selected from the group consisting of a promoter sequence, signals for initiation of translation, signals for termination of translation, and appropriate regions for regulation of transcription. In certain embodiments, the vectors can be stably maintained in the host cell and can, optionally, contain signal sequences directing the secretion of translated protein. Other embodiments provide vectors that are not stable in transformed host cells. Vectors can integrate into the host genome or be autonomously-replicating vectors.

In a specific embodiment, a vector comprises a promoter operably linked to a PKC inhibitor encoding nucleic acid sequence, one or more origins of replication, and, optionally, one or more selectable markers (e.g., an antibiotic resistance gene). Non-limiting exemplary vectors for the expression of the polypeptides of the invention include pBr-type vectors, pET-type plasmid vectors (PROMEGA), pBAD plasmid vectors (INVITROGEN), and pVAX plasmid vectors (INVITROGEN), or others provided in the examples below. Furthermore, vectors according to the invention are useful for transforming host cells for the cloning or expression of the nucleotide sequences of the invention.

Promoters which may be used to control expression include, but are not limited to, the CMV promoter, the SV40 early promoter region (Bernoist and Chambon Nature 290:304-310, 1981), the promoter contained in the 3′ long terminal repeat of Rous sarcoma virus (Yamamoto, et al. Cell 22:787-797, 1980), the herpes thymidine kinase promoter (Wagner et al. Proc. Natl. Acad. Sci. USA 78:1441-1445, 1981), the regulatory sequences of the metallothionein gene (Brinster et al. Nature 296:39-42, 1982); prokaryotic vectors containing promoters such as the β-lactamase promoter (Villa-Kamaroff, et al. Proc. Natl. Acad. Sci. USA 75:3727-3731, 1978), or the tac promoter (DeBoer, et al. Proc. Natl. Acad. Sci. USA 80:21-25, 1983); the lung specific promoters such as surfactant protein B promoter (Venkatesh et al., Am. J. Physiol. 268 (Lung Cell Mol. Physiol. 12):L674-L682, 1995); see also, “Useful Proteins from Recombinant Bacteria” in Scientific American, 1980, 242:74-94; plant expression vectors comprising the nopaline synthetase promoter region (Herrera-Estrella et al. Nature 303:209-213, 1983) or the cauliflower mosaic virus 35S RNA promoter (Gardner, et al. Nucl. Acids Res. 9:2871, 1981), and the promoter of the photosynthetic enzyme ribulose biphosphate carboxylase (Herrera-Estrella et al. Nature 310:115-120, 1984); promoter elements from yeast or fungi such as the Gal 4 promoter, the ADC (alcohol dehydrogenase) promoter, PGK (phosphoglycerol kinase) promoter, and/or the alkaline phosphatase promoter.

Nucleotide sequences encoding polypeptides with enhanced PKC inhibitory activity can be obtained by “gene shuffling” (also referred to as “directed evolution”, and “directed mutagenesis”), and used in the compositions and methods of the present invention. Gene shuffling is a process of randomly recombining different sequences of functional genes (recombining favorable mutations in a random fashion) (U.S. Pat. Nos. 5,605,793; 5,811,238; 5,830,721; and 5,837,458). Thus, protein engineering can be accomplished by gene shuffling, random complex permutation sampling, or by rational design based on three-dimensional structure and classical protein chemistry (Cramer et al., Nature, 391:288-291, 1998; and Wulff et al., The Plant Cell, 13:255-272, 2001).

The invention also provides host cells transformed by a polynucleotide encoding a PKC inhibitor and the production of the PKC inhibitor by the transformed host cells. Transformed host cells according to the invention are cultured under conditions allowing the replication and/or the expression of the nucleotide sequence encoding the PKC inhibitor. PKC inhibitory polypeptides are recovered from culture media and purified, for further use, according to methods known in the art.

The host cell may be chosen from eukaryotic or prokaryotic systems, for example bacterial cells (Gram negative or Gram positive), yeast cells, animal cells, human cells, plant cells, and/or insect cells using baculovirus vectors. In some embodiments, the host cell for expression of the polypeptides include, and are not limited to, those taught in U.S. Pat. Nos. 6,319,691; 6,277,375; 5,643,570; 5,565,335; Unger The Scientist 11(17):20, 1997; or Smith The Scientist 12(22):20, 1998, each of which is incorporated by reference in its entirety, including all references cited within each respective patent or reference. Other exemplary, and non-limiting, host cells include Staphylococcus spp., Enterococcus spp., E. coli, and Bacillus subtilis; fungal cells, such as Streptomyces spp., Aspergillus spp., S. cerevisiae, Schizosaccharomyces pombe, Pichia pastoris, Hansela polymorphs, Kluveromyces lactis, and Yarrowia lipolytica; insect cells such as Drosophila S2 and Spodoptera Sf9 cells; animal cells such as CHO, COS, HeLa, C127, 3T3, BHK, 293 and Bowes melanoma cells; and plant cells. A great variety of expression systems can be used to produce the PKC inhibitory polypeptides and encoding polynucleotides can be modified according to methods known in the art to provide optimal codon usage for expression in a particular expression system.

Furthermore, a host cell strain may be chosen that modulates the expression of the inserted sequences, modifies the gene product, and/or processes the gene product in the specific fashion. Expression from certain promoters can be elevated in the presence of certain inducers; thus, expression of the genetically engineered polypeptide may be controlled. Furthermore, different host cells have characteristic and specific mechanisms for the translational and post-translational processing and modification (e.g., glycosylation, phosphorylation) of proteins. Appropriate cell lines or host systems can be chosen to ensure the desired modification and processing of the foreign protein expressed. For example, expression in a bacterial system can be used to produce an unglycosylated core protein product whereas expression in yeast will produce a glycosylated product. Expression in mammalian cells can be used to provide “native” glycosylation of a heterologous protein. Furthermore, different vector/host expression systems may effect processing reactions to different extents.

Nucleic acids and/or vectors encoding PKC inhibitory polypeptides can be introduced into host cells by well-known methods, such as, calcium phosphate transfection, DEAE-dextran mediated transfection, transfection, microinjection, cationic lipid-mediated transfection, electroporation, transduction, scrape loading, ballistic introduction and infection (see, for example, Sambrook et al. [1989] Molecular Cloning: A Laboratory Manual, 2^{nd}Ed., Cold Spring Harbor Laboratory Press, Cold Spring Harbor, N.Y.).

In the context of the instant invention, the terms “polypeptide”, “peptide” and “protein” are used interchangeably to refer to an amino acid sequence of any length unless otherwise specified.

PKC inhibitory polypeptides used in the compositions and methods of the present invention may further contain linkers that facilitate the attachment of the fragments to a carrier molecule for delivery or diagnostic purposes. The linkers can also be used to attach fragments according to the invention to solid support matrices for use in affinity purification protocols. In this aspect of the invention, the linkers specifically exclude, and are not to be considered anticipated, where the fragment is a subsequence of another peptide, polypeptide, or protein as identified in a search of protein sequence databases as indicated in the preceding paragraph. In other words, the non-identical portions of the other peptide, polypeptide, or protein is not considered to be a “linker” in this aspect of the invention. Non-limiting examples of “linkers” suitable for the practice of the invention include chemical linkers (such as those sold by Pierce, Rockford, Ill.), peptides that allow for the connection of the immunogenic fragment to a carrier molecule (see, for example, linkers disclosed in U.S. Pat. Nos. 6,121,424; 5,843,464; 5,750,352; and 5,990,275, hereby incorporated by reference in their entirety). In various embodiments, the linkers can be up to 50 amino acids in length, up to 40 amino acids in length, up to 30 amino acids in length, up to 20 amino acids in length, up to 10 amino acids in length, or up to 5 amino acids in length.

In other specific embodiments, the PKC inhibitory polypeptide may be expressed as a fusion, or chimeric protein product (comprising the PKC inhibitory polypeptide joined via a peptide bond to a heterologous protein sequence (e.g., a different protein)). Such a chimeric product can be made by ligating the appropriate nucleic acid sequences encoding the desired amino acid sequences to each other by methods known in the art, in the proper coding frame, and expressing the chimeric product by methods commonly known in the art (see, for example, U.S. Pat. No. 6,342,362, hereby incorporated by reference in its entirety; Altendorf et al. J. of Experimental Biology 203:19-28, 1999-WWW, 2000; Baneyx Biotechnology 10:411-21, 1999; Eihauer et al. J. Biochem Biophys Methods 49:455-65, 2001; Jones et al. J. of Chromatography A. 707:3-22, 1995; Margolin Methods 20:62-72, 2000; Puig et al. Methods 24:218-29, 2001; Sassenfeld TibTech 8:88-93, 1990; Sheibani Prep. Biochem. & Biotechnol. 29(1):77-90, 1999; Skerra et al. Biomolecular Engineering 16:79-86, 1999; Smith The Scientist 12(22):20, 1998; Smyth et al. Methods in Molecular Biology, 139:49-57, 2000; Unger The Scientist 11(17):20, 1997, each of which is hereby incorporated by reference in their entireties). Alternatively, such a chimeric product may be made by protein synthetic techniques, e.g., by use of a peptide synthesizer. Fusion peptides can comprise PKC inhibitory polypeptides and one or more protein transduction domains, as described above. Such fusion peptides are particularly useful for delivering the cargo polypeptide through the cell membrane.

Decreasing the amount of PKC enzymatic activity within a tissue is useful in preventing an RSV infection, or in treating an existing RSV infection. Thus, according to the methods of the subject invention, the amount of PKC activity can be decreased within a tissue by directly administering the PKC inhibitor to a patient suffering from or susceptible to an RSV infection (such as exogenous delivery of a PKC inhibitory polypeptide or other compound exhibiting PKC inhibitory activity) or by indirect or genetic means (such as delivery of a nucleotide sequence that interferes with expression of PKC at the transcriptional or translational level, or otherwise down-regulating the endogenous PKC enzymatic activity).

As used herein, the term “administration” or “administering” refers to the process of delivering an agent to a patient, wherein the agent directly or indirectly decreases PKC enzymatic function within the patient and, preferably, at the target site, such as bronchial epithelium. The process of administration can be varied, depending on the agent, or agents, and the desired effect. Thus, wherein the agent is genetic material, such as DNA, the process of administration involves administering the interfering DNA, or the DNA encoding a PKC inhibitory polypeptide, to a patient in need of such treatment. Administration can be accomplished by any means appropriate for the therapeutic agent, for example, by parenteral, mucosal, pulmonary, topical, catheter-based, or oral means of delivery. Parenteral delivery can include for example, subcutaneous intravenous, intramuscular, intra-arterial, and injection into the tissue of an organ, particularly tumor tissue. Mucosal delivery can include, for example, intranasal delivery. According to the method of the present invention, a PKC inhibitor is preferably administered into the airways of a patient, i.e., nose, sinus, throat, lung, for example, as nose drops, by nebulization, vaporization, or other methods known in the art. Oral or intranasal delivery can include the administration of a propellant. Pulmonary delivery can include inhalation of the agent. Catheter-based delivery can include delivery by iontropheretic catheter-based delivery. Oral delivery can include delivery of a coated pill, or administration of a liquid by mouth. Administration can generally also include delivery with a pharmaceutically acceptable carrier, such as, for example, a buffer, a polypeptide, a peptide, a polysaccharide conjugate, a liposome, and/or a lipid. Gene therapy protocol is also considered an administration in which the therapeutic agent is a polynucleotide capable of accomplishing a therapeutic goal when expressed as a transcript or a polypeptide into the patient. Further information concerning applicable gene therapy protocols is provided in the examples disclosed herein.

The pharmaceutical compositions of the subject invention can be formulated according to known methods for preparing pharmaceutically useful compositions. Formulations are described in a number of sources which are well known and readily available to those skilled in the art. For example, Remington's Pharmaceutical Science (Martin E W [1995] Easton, Pa., Mack Publishing Company, 19^{th} ed.) describes formulations which can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

Therapeutically effective and optimal dosage ranges for PKC inhibitors can be determined using methods known in the art. The specific dosage appropriate for administration is readily determined by one of ordinary skill in the art according to the factors discussed above (see, for example, Remington's Pharmaceutical Sciences). In addition, the estimates for appropriate dosages in humans may be extrapolated from determinations of the level of PKC inhibitory activity determined in vitro and/or the amount of PKC antagonist effective in inhibiting RSV infection in an animal model. Guidance as to appropriate dosages to achieve an anti-viral effect is provided from the exemplified assays disclosed herein.

Because PKC is an intracellular protein, preferred embodiments of the invention involve using pharmaceutically acceptable inhibitor formulations capable of permeating the plasma membrane. Small, apolar molecules are often membrane permeable. The membrane permeability of other molecules can be enhanced by a variety of methods known to those of skill in the art, including dissolving them in hypotonic solutions, coupling them to transport proteins, and packaging them in micelles. As indicated above, PKC inhibitory peptides can be modified by covalently incorporating myristoyl-moieties, translocating-peptides (such as HIV-1-Tat), or peptides containing basic amino acid residues, such as arginine. These modifications allow the peptides to pass through the plasma membrane and enter into the cells. In addition, plasmids encoding PKC regulatory domains or siRNAs complexed with nanoparticles targeting specific cell types (such as bronchial epithelium) can be used.

The present invention further provides methods of making the host cells, pharmaceutical compositions, and vectors described herein by combining the various components using methods known in the art.

The methods of the present invention can further comprise administering one or more additional anti-viral agents to the patient, which are effective at inhibiting infection by RSV or other viruses. The compositions of the present invention can further comprise such additional anti-viral agents. In addition to PKC inhibitors, such as pseudosubstrate sequences, inhibitors targeting viral replication and infection are considered compatible. For example, it is reported in the literature that Ribavirin is used for targeting viral replication. Other anti-RSV agents can be used with the methods, compositions, vectors, and host cells of the present invention. For example, Synagis, a monoclonal antibody preparation, blocks RSV fusion. In the same way, different chemical compounds targeting RSV binding and fusion, such as the biphenyl analog RFI-641 and the synthetic peptide containing amino acids 77 to 95 of the intracellular GTPase RhoA, can be utilized. This latter peptide disrupts F or G binding to cellular glycosaminoglycans or other receptors because of charge-charge interactions. Furthermore, caveolae formation can be targeted by the use of caveolin scaffolding domain peptides (e.g., a.a. sequence: DGIWKASFTTFTVTKYWFYR (SEQ ID NO. 1)), which can be modified to allow them to enter into the cells. Cholesterol-depleting compounds, such as lovastatin, can also be used as antiviral agents in conjunction with the present invention. These and other approaches can be used in conjunction with the strategy of the present invention, which involves decreasing PKC activity and, consequently, inhibiting RSV infection (e.g., by blocking RSV fusion).

The term “patient”, as used herein, refers to any vertebrate species. Preferably, the patient is of a mammalian species. Mammalian species which benefit from the disclosed methods of treatment include, and are not limited to, primates, such as humans, apes, chimpanzees, orangutans, and monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. Human or non-human animal patients can range in age from neonates to elderly. The nucleotide sequences and polypeptides can be administered to patients of the same species or from different species. For example, mammalian homologs can be administered to human patients.

As used herein, the terms “comprising”, “consisting of”, and “consisting essentially of” are defined according to their standard meaning and may be substituted for one another throughout the instant application in order to attach the specific meaning associated with each term.

As used herein, the phrase “inhibiting RSV infection” means preventing or reducing the rate of infection of cells by RSV in vitro or in vivo, or preventing or alleviating one or more symptoms associated with RSV infection in a human or animal patient.

As used herein, the term “protein kinase C” or “PKC” refers to an enzyme that facilitates phosphorylation of serine and threonine residues in a variety of proteins. PKC is a multigene family of phospholipid-dependent, serine-threonine kinases central to many signal transduction pathways. Molecular cloning studies have identified ten members of the PKC family. These family members, called isozymes, are encoded by nine different genes. The ten isozymes are designated as the alpha, beta I, beta II, gamma, delta, epsilon, zeta, eta, l/lambda and theta isozymes (Y. Nishizuka, Science 258, 607-614 (1992); L. A. Selbie, C. Schmitz-Peiffer, Y. Sheng, T. J. Biden, J. Biol. Chem. 268, 24296-24302 (1993)). Based on sequence homology and biochemical properties, the PKC gene family has been divided into three groups: (i) the “conventional” PKCs, the alpha, beta I, beta II, and gamma isozymes, are regulated by calcium, diacylglycerol and phorbol esters; (ii) the “novel” PKCs, the delta, epsilon, theta and eta isozymes, are calcium-independent, but diacylglycerol- and phorbol ester-sensitive; and (iii) the “atypical” PKCs, the zeta and l/lambda.isozymes, are insensitive to calcium, diacylglycerol and phorbol 12-myristate 13-acetate. In addition, two related phospholipid-dependent kinases, PKC M and protein kinase D, share sequence homology in their regulatory domains to novel PKCs and may constitute a subgroup (F. J. Johannes, J. Prestle, S. Eis, P. Oberhagemann, K. Pfizenmaier, Biol. Chem. 269, 6140-6148, 1994; A. M. Valverde, J. Sinnett-Smith, J. Van Lint, E. Rozengurt, Proc. Natl. Acad. Sci. USA 91, 8572-8576, 1994). Unless specified, the terms “protein kinase C” or “PKC” are intended to refer to one or more isoforms (e.g., alpha, beta I, beta II, gamma, delta, epsilon, zeta, eta, l/lambda and theta) of the enzyme, such as PKC alpha.

As used herein, the term “protein kinase C activity” or “PKC activity”, refers to the normal functions of PKC, many of which are activation-dependent, such as the phosphorylation of substrates (i.e., the catalytic activity of PKC), autophosphorylation, movement from one intracellular location to another upon activation (i.e., intracellular translocation), and binding to or release from one or more proteins that anchor PKC in a given location.

As used herein, the term “protein kinase C inhibitor” or “PKC inhibitor” refers to any agent or treatment capable of decreasing the normal endogenous level of PKC activity within a patient. An agent or treatment inhibits the activity of PKC if it affects (1) one or more of the normal functions of PKC, or (2) the expression, modification, regulation, activation or degradation of PKC or a molecule acting upstream of PKC in a regulatory or enzymatic pathway. The inhibitor decreases the normal endogenous level of PKC activity of the patient to which the inhibitor is administered. For example, where the patient is human, an inhibitor decreasing the normal endogenous level of human PKC activity is administered. Optionally, the PKC inhibitor used in the methods and composition of the present invention is selective for one or of the PKC isozymes, such as PKC alpha.

### EXAMPLE 1

### Requirement of Different Signaling Elements for Successful RSV Infection in Primary NHBE Cells

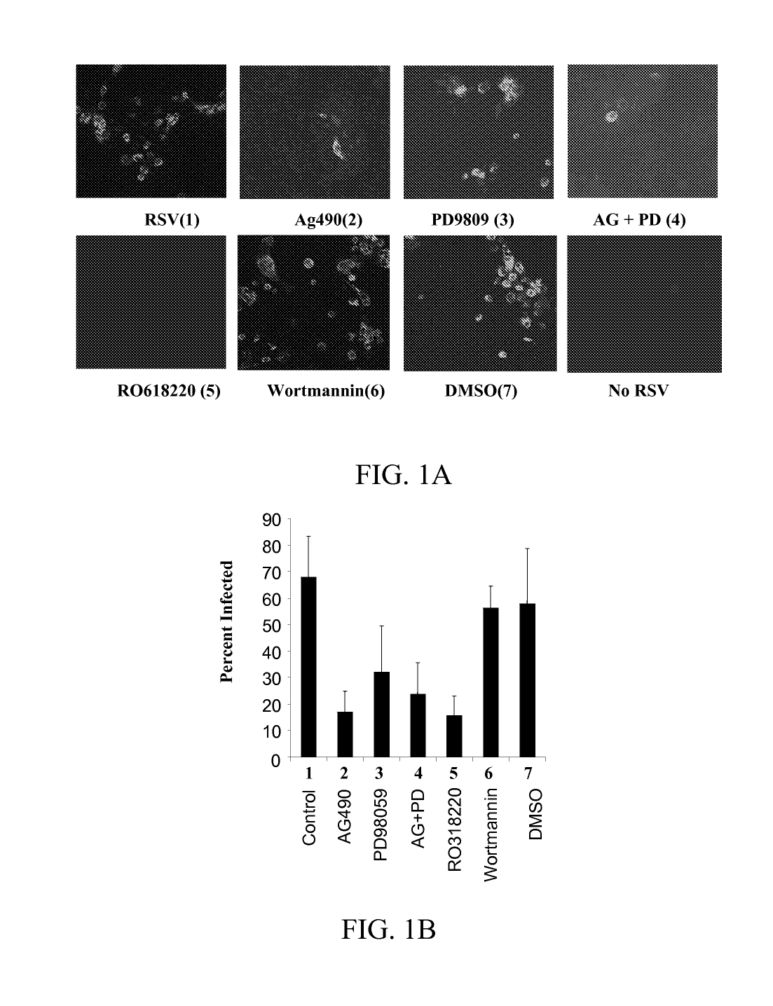

To determine if different signaling molecules related to the ERK pathway are required for a successful RSV infection, primary NHBE cells were exposed to various inhibitors previously to being infected with a sucrose-purified RSV preparation. Exposure of NHBE cells to AG490, PD98059, and Ro318220 caused a significant reduction in the number of infected cells, while Wortmannin did not have an effect on viral replication, as shown in FIGS. 1A and 1B. These results strongly suggest that JAK, ERK-1/2, and PKC, but not PI-3K, are required for a successful RSV infection in bronchial epithelial cells. The fact that the highest reduction in percentage of infected cells was seen with PKC inhibitor suggests that initial events following RSV exposure may involve PKC activation. A previous report implicated PKCζ in the early stages of RSV infection in A549 cells and suggested that it may be responsible for activating ERK-2 (Monick, M. et al., J Immunol, 166(4):2681-2687, 2001). Also, other PKC isoforms are activated later during the infection, which could potentially play a role in the late phase of ERK activation (Monick, M. et al., J Immunol, 166(4):2681-2687, 2001).

### EXAMPLE 2

### PKC Inhibitors Block RSV Infection

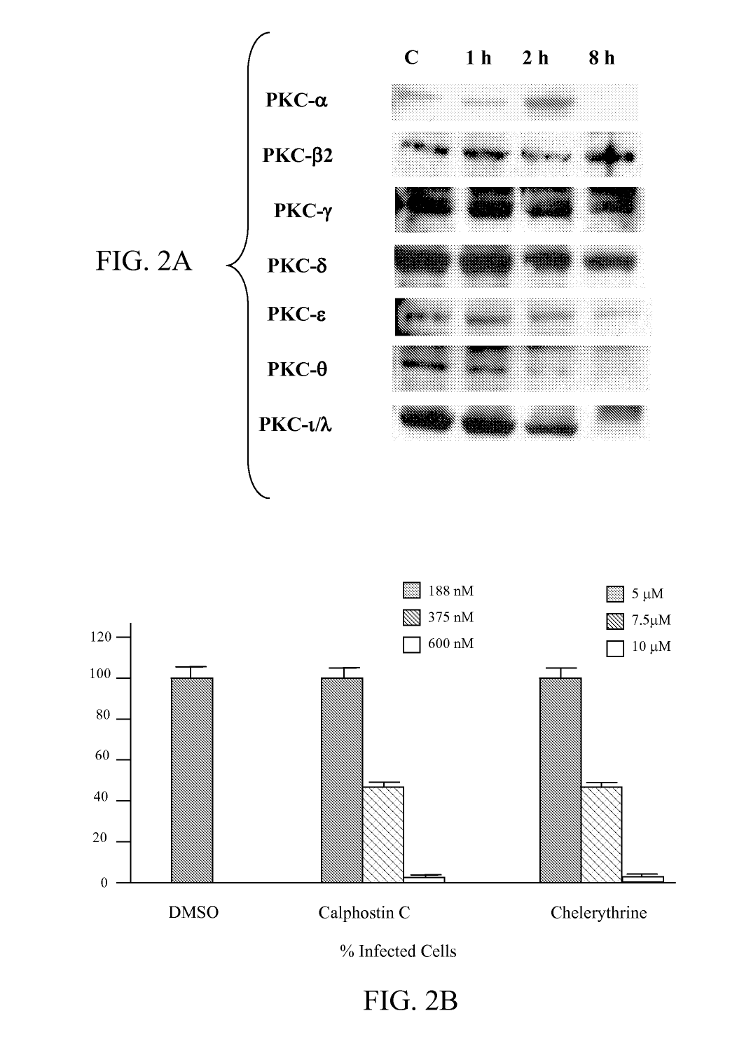

A previous report indicated that several PKC isozymes are activated at early and late stages of RSV infection in A549 cells, there is no report if any of the PKC isozymes is required for an efficient RSV infection. The possibility whether PKCs are involved in normal human epithelial cells was tested in cultures of primary cells, normal human bronchial epithelial cells. Results show that NHBE cells express PKC-α, β2, γ, δ, ε, ↓, ι, and λ (FIG. 2A) and a time course assay demonstrated that RSV infection caused changes in the levels of different PKC isozymes at different time points. Such changes are reflected in the reduction of the expression of these PKC isoforms, suggesting the previous activation of these isozymes. Moreover, PKC inhibitors, Calphostin C, and Chelerythrine reduced in a dose-dependent manner the number of infected cells (FIG. 2B) in which 50% inhibition was reached at concentrations of 375 nM for Calphostin C and 7.5 μM for Chelerythrine. Because Calphostin C is considered an inhibitor of classical and novel PKC isozymes, a myristoylated PKC-α/β pseudosubstrate peptide (the myristoylated moiety allows the peptide to enter into the cells) was used to determine if the classical isozymes are involved in RSV infection (N-Myr-Phe-Ala-Arg-Lys-Gly-Ala-Leu-Arg-Gln) (SEQ ID NO: 3). The myristoylated portion (Myr) is at the N-terminal end of the sequence peptide above. This sequence was obtained from the pseudosubstrate sequence (a.a. 20-28) of PKC-alpha and beta. The peptide's molecular weight is 1,255.6 Da. and it is soluble in water.

As shown in single cell fluorescent assays (FIG. 2B), the incubation of NHBE cells with myristoylated PKC-α/β pseudosubstrate peptide previous to being exposed to RSV at an infectious dose of 1 MOI reduced the number of infected cells in a dose-responsive way. The numbers of infected cells dramatically drop when they were exposed to a pseudosubstrate inhibitor concentration of 25 04. Previous studies have reported that the pseudosubstrate peptide inhibits 100% of the PKC activity at 50 04.

As it is demonstrated using FACS analysis, a non-myristoylated peptide with the same pseudosubstrate amino acid sequence did not block RSV infection (FIG. 2B), which indicates that the peptide did not interfere with RSV binding to the cell. Overall, these results indicate that the activation of PKC is playing a role in RSV infection.

### EXAMPLE 3

### PKC-α Activation and Its Translocation to Cell Membrane Induced by RSV

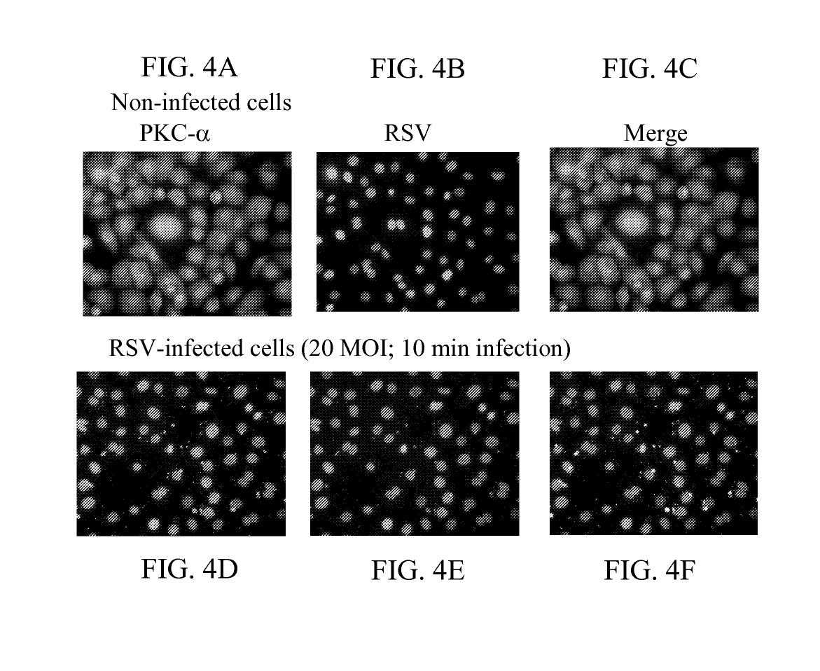

To determine the location and phosphorylation status of PKC-α by immunocytofluorescence and confocal microscopy, NHBE cells were exposed to RSV at an infectious dose of 20 MOI. PKC-α was first studied because of the role that this isozyme plays during the formation of the caveolae, which has been indicated as a required system for both RSV infection and maturation. PKC-α translocates from the cytoplasm to the cell plasma membrane and colocalizes with viral particles as early as 10 minutes after exposure to RSV (FIGS. 4A-4F). PKC-α colocalizes with the viral particles up to 1 hr at the cell membrane. Whether the persistence of co-localization is due to the binding of new viral particles to the cells or the formation of a stable complex is unknown at the present time. Several studies have demonstrated that autophosphorylated PKC-α migrates to the cell membrane for further signaling events. There are four potential phosphorylation sites in PKC-α, Thr-250, Thr-497, Thr-638, and Ser-657, which are phosphorylated in activated PKC-α. The phosphorylation status of the translocated PKC-α was determined by using an anti-phospho Thr-638 PKC-α antibody. Confocal images (FIGS. 4A-4F) showed an increase of phospho-PKC-α which, in addition, is associated with those viral particles contacting the cells as early as 10 minutes after RSV exposure. Such co-localization signal at the cell membrane is still present 1 hour after virus exposure. In addition, viral particles are required for the activation of PKC-α as there was no increase in phosphorylation of PKC-α when NHBE cells were exposed to a sham treatment, which is the filtrate resulting of centrifuging RSV suspension through Centricon YM-100. When PKC-α pseudosubstrate peptide was used at 50 μM, there was an expected reduction of phospho-PKC-α. Surprisingly, though, there was also an apparent reduction in the number of RSV particles contacting the cells. Overall, these results indicate that RSV particles induce translocation and activation of PKC-α when contacting NHBE cells.

### EXAMPLE 4

### PKC-α Activation is Required RSV Fusion

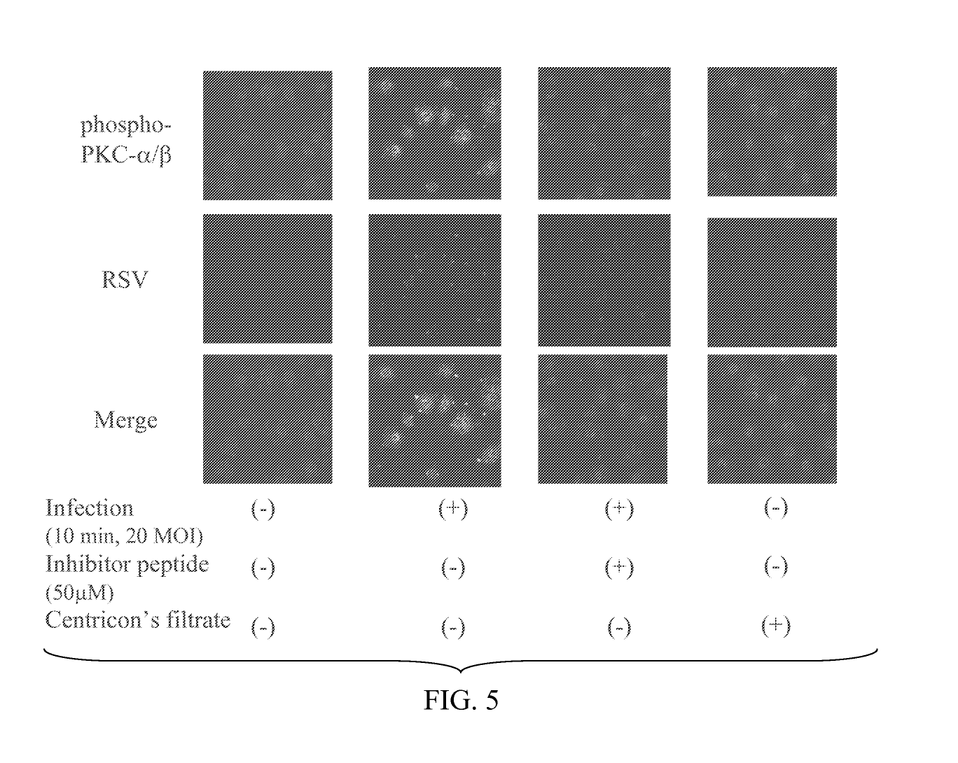

Because the PKC-α/β pseudosubstrate inhibitor caused an apparent reduction in the number of viral particles contacting NHBE cells, the present inventor hypothesized that an early event of RSV infection is compromised when PKC-α activity is inhibited. A fluorescence microscopy assay based on a fluorescence-dequenching method previously described was used to determine if PKC-α activity inhibition prevents fusion of RSV with NHBE cells. In this approach, RSV is labeled with octadecyl-rhodamine R18 at self-quenching concentration, and the viral fusion with unlabeled NHBE cells is directly observed in a fluorescence microscopy as an increase in quantum yield of R18 due to membrane fusion events and the resulting dilution of dye in the merged membrane. As shown in FIG. 5, PKC-α/β pseudosubstrate peptide impairs RSV fusion with NHBE cells. Moreover, as it was paralleled in single cell fluorescent assays, RSV fusion was significantly inhibited when NHBE cells were pre-treated with PKC-α/β pseudosubstrate peptide at 25 μM; and, practically absent when cells were pre-treated with peptide inhibitor at 50 μM. Thus, PKC-α activity is required during RSV fusion to NHBE cells.

### EXAMPLE 5

### PKC-α Activity Inhibition Impairs RSV Infection by Affecting RhoA Location in the Cell

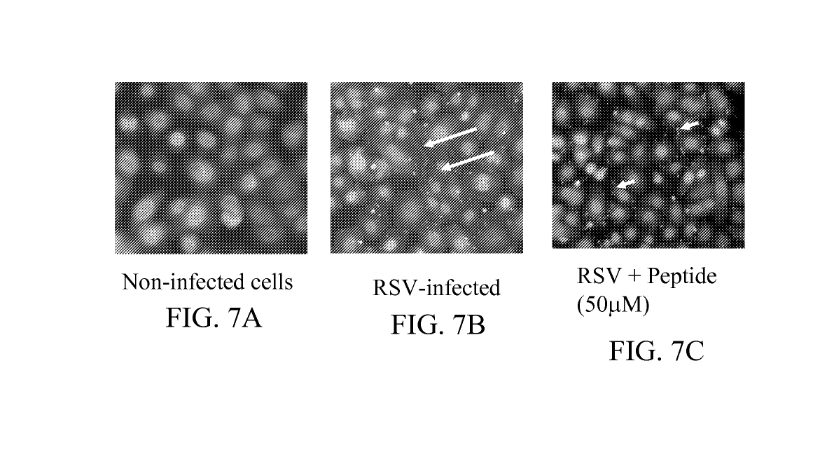

Previous reports have highlighted the role of RhoA during RSV infection. RhoA have been indicated as required for RSV fusion. A RhoA peptide constructed from the RhoA primary sequence to which RSV F binds to impairs RSV infection both in vivo and in vitro. However, it is unknown how RhoA is recruited to the place which RSV contacts the cell. The present inventor hypothesized that PKC-α activity is required for a proper location of RhoA at the cell membrane to serve as potential anchor for RSV-F protein. As it is shown in FIG. 7A, RhoA is predominantly located at the cell cytoplasm in non-infected cells. After 10 minutes of RSV exposure at an infectious dose of 20 MOI, RhoA is translocated at the cell plasma membrane. However, RhoA is sequestered in a very restricted location when NHBE cells are incubated with PKC-α/β pseudosubstrate peptide (50 μM) before being infected with RSV, as shown in FIG. 7C. Thus, these results suggest that PKC-α activity is required for a proper location of RhoA at the cell membrane for successful RSV infection.

### EXAMPLE 6

### Gene Therapy

In the therapeutic and prophylactic methods of the present invention, the nucleotide sequence encoding the PKC inhibitor can be administered to a patient in various ways. It should be noted that the nucleotide sequence can be administered alone or as an active ingredient in combination with pharmaceutically acceptable carriers, diluents, adjuvants and vehicles. Preferably, the nucleotide sequence is administered intranasally, bronchially, via inhalation pathways, for example. The patient being treated is a warm-blooded animal and, in particular, mammals including humans. The pharmaceutically acceptable carriers, diluents, adjuvants and vehicles as well as implant carriers generally refer to inert, non-toxic solid or liquid fillers, diluents or encapsulating material not reacting with the active ingredients of the present invention.

It is noted that humans are treated generally longer than the mice exemplified herein, which treatment has a length proportional to the length of the disease process and drug effectiveness. The doses may be single doses or multiple doses over a period of several days, but single doses are preferred.

The carrier for gene therapy can be a solvent or dispersing medium containing, for example, water, ethanol, polyol (for example, glycerol, propylene glycol, liquid polyethylene glycol, and the like), suitable mixtures thereof, and vegetable oils.

Proper fluidity, when desired, can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives that enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Examples of delivery systems useful in the present invention include, but are not limited to: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other delivery systems and modules are well known to those skilled in the art.

A pharmacological formulation of the nucleotide sequence utilized in the present invention can be administered orally to the patient. Conventional methods such as administering the compounds in tablets, suspensions, solutions, emulsions, capsules, powders, syrups and the like are usable. Known techniques which deliver the vaccine orally or intravenously and retain the biological activity are preferred.

In one embodiment, the nucleotide sequence can be administered initially by nasal infection to decrease the local levels of PKC enzymatic activity. The patient's PKC activity levels are then maintained at a diminished level by an oral dosage form, although other forms of administration, dependent upon the patient's condition and as indicated above, can be used. The quantity of nucleotide molecule to be administered will vary for the patient being treated and will vary from about 100 ng/kg of body weight to 100 mg/kg of body weight per day and preferably will be from 10 mg/kg to 10 mg/kg per day.

As indicated above, standard molecular biology techniques known in the art and not specifically described can be generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al. (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659; and 5,272,057, the contents of which are incorporated herein by reference in their entirety. Polymerase chain reaction (PCR) can be carried out generally as in PCR Protocols: A Guide To Methods And Applications, Academic Press, San Diego, Calif (1990). In-situ (In-cell) PCR in combination with Flow Cytometry can be used for detection of cells containing specific DNA and mRNA sequences (Testoni et al., 1996, Blood 87:3822).

As used herein, the term “gene therapy” refers to the transfer of genetic material (e.g., DNA or RNA) of interest into a host to treat or prevent a genetic or acquired disease or condition phenotype. The genetic material of interest encodes a product (e.g., a protein, polypeptide, peptide or functional RNA) whose production in vivo is desired. For example, in addition to the nucleotide encoding the PKC inhibitor, the genetic material of interest can encode a hormone, receptor, or other enzyme, polypeptide or peptide of therapeutic value. For a review see, in general, the text “Gene Therapy” (Advances in Pharmacology 40, Academic Press, 1997).

Two basic approaches to gene therapy have evolved: (1) ex vivo and (2) in vivo gene therapy. In ex vivo gene therapy, cells are removed from a patient, and while being cultured are treated in vitro. Generally, a functional replacement gene is introduced into the cell via an appropriate gene delivery vehicle/method (transfection, transduction, homologous recombination, etc.) and an expression system as needed and then the genetically modified cells are expanded in culture and returned to the host/patient. These genetically reimplanted cells produce the transfected gene product in situ. Alternatively, a xenogenic or allogeneic donor's cells can be genetically modified with the nucleotide sequence in vitro and subsequently administered to the patient.

In in vivo gene therapy, target cells are not removed from the patient; rather, the gene to be transferred is introduced into the cells of the recipient organism in situ, that is within the recipient. Alternatively, if the host gene is defective, the gene is repaired in situ. These genetically modified cells produce the transfected gene product in situ.

The gene expression vehicle is capable of delivery/transfer of heterologous nucleic acids into a host cell. As indicated previously, the expression vehicle may include elements to control targeting, expression and transcription of the nucleotide sequence in a cell selective or tissue-specific manner, as is known in the art. It should be noted that often the 5′UTR and/or 3′UTR of the gene may be replaced by the 5′UTR and/or 3′UTR of the expression vehicle. Therefore as used herein the expression vehicle may, as needed, not include the 5′UTR and/or 3′UTR and only include the specific amino acid coding region.

The expression vehicle can include a promoter for controlling transcription of the heterologous material and can be either a constitutive or inducible promoter to allow selective transcription. Enhancers that may be required to obtain necessary transcription levels can optionally be included. Enhancers are generally any non-translated DNA sequence which works contiguously with the coding sequence (in cis) to change the basal transcription level dictated by the promoter. The expression vehicle can also include a selection gene as described herein below.

Vectors can be introduced into cells or tissues by any one of a variety of known methods within the art. Such methods can be found generally described in Sambrook et al., Molecular Cloning: A Laboratory Manual, Cold Springs Harbor Laboratory, New York (1989, 1992); in Ausubel et al., Current Protocols in Molecular Biology, John Wiley and Sons, Baltimore, Md. (1989); Chang et al., Somatic Gene Therapy, CRC Press, Ann Arbor, Mich. (1995); Vega et al., Gene Targeting, CRC Press, Ann Arbor, Mich. (1995); Vectors: A Survey of Molecular Cloning Vectors and Their Uses, Butterworths, Boston, Mass. (1988); and Gilboa et al. (1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of a DNA viral vector for introducing and expressing recombinant nucleotide sequences is the adenovirus derived vector Adenop53TK. This vector expresses a herpes virus thymidine kinase (TK) gene for either positive or negative selection and an expression cassette for desired recombinant sequences. This vector can be used to infect cells that have an adenovirus receptor which includes most cancers of epithelial origin as well as others. This vector as well as others that exhibit similar desired functions can be used to treat a mixed population of cells and can include, for example, an in vitro or ex vivo culture of cells, a tissue or a human subject.

Additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotic. Such protection ensures that if, for example, mutations arise that produce altered forms of the viral vector or recombinant sequence, cellular transformation will not occur. Features that limit expression to particular cell types or tissue types can also be included. Such features include, for example, promoter and regulatory elements that are specific for the desired cell type or tissue type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods of the present invention will depend on desired the cell type or cell types to be targeted and will be known to those skilled in the art. For example, if RSV infection is to be inhibited (i.e., treated or prevented), then a vector specific for such respiratory mucosal epithelial cells would preferably be used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles that are capable of undergoing further rounds of infection. The vector's genome is also engineered to encode and express the desired recombinant nucleotide sequence. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector will depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment, administration can also be performed by, for example, intravenous or subcutaneous injection into the subject. Injection of the viral vectors into a spinal fluid can also be used as a mode of administration, especially in the case of RNA virus infections of the central nervous system. Following injection, the viral vectors will circulate until they recognize host cells with the appropriate target specificity for infection.

An alternate mode of administration can be by direct inoculation locally at the site of the disease or pathological condition or by inoculation into the vascular system supplying the site with nutrients or into the spinal fluid. Local administration is advantageous because there is no dilution effect and, therefore, a smaller dose is required to achieve expression in a majority of the targeted cells. Additionally, local inoculation can alleviate the targeting requirement required with other forms of administration since a vector can be used that infects all cells in the inoculated area. If expression is desired in only a specific subset of cells within the inoculated area, then promoter and regulatory elements that are specific for the desired subset can be used to accomplish this goal. Such non-targeting vectors can be, for example, viral vectors, viral genome, plasmids, phagemids and the like. Transfection vehicles such as liposomes and colloidal polymeric particles can also be used to introduce the non-viral vectors described above into recipient cells within the inoculated area. Such transfection vehicles are known to those skilled within the art.

Direct DNA inoculations can be administered as a method of vaccination. Plasmid DNAs encoding influenza virus hemagglutinin glycoproteins have been tested for the ability to provide protection against lethal influenza challenges. In immunization trials using inoculations of purified DNA in saline, 67-95% of test mice and 25-63% of test chickens were protected against the lethal challenge. Good protection was achieved by intramuscular, intravenous and intradermal injections. In mice, 95% protection was achieved by gene gun delivery of 250-2500 times less DNA than the saline inoculations. Successful DNA vaccination by multiple routes of inoculation and the high efficiency of gene-gun delivery highlight the potential of this promising new approach to immunization. Plasmid DNAs expressing influenza virus hemagglutinin glycoproteins have been tested for their ability to raise protective immunity against lethal influenza challenges of the same subtype. In trials using two inoculations of from 50 to 300 micrograms of purified DNA in saline, 67-95% of test mice and 25-63% of test chickens have been protected against a lethal influenza challenge. Parenteral routes of inoculation that achieve good protection include intramuscular and intravenous injections. Successful mucosal routes of vaccination included DNA drops administered to the nares or trachea. By far, the most efficient DNA immunizations were achieved by using a gene gun to deliver DNA-coated gold beads to the epidermis. In mice, 95% protection was achieved by two immunizations with beads loaded with as little as 0.4 micrograms of DNA. The breadth of routes supporting successful DNA immunizations, coupled with the very small amounts of DNA required for gene-gun immunizations, highlight the potential of this remarkably simple technique for the development of subunit vaccines. In contrast to the DNA based antigen vaccines, the present invention provides the development of an intranasal gene transfer method using a PKC inhibitor, which can be used as a prophylaxis or treatment against RSV.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A method of inhibiting a respiratory syncytial virus (RSV) infection in a mammal by decreasing the endogenous protein kinase C (PKC) activity within the mammal.

2. The method of claim 1, wherein the PKC activity is that of at least one classical PKC isoform.

3. The method of claim 1, wherein said decreasing comprises administering at least one PKC inhibitor to the mammal.

4. The method of claim 3, wherein the at least one PKC inhibitor is selected from the group consisting of AG 490, PD98059, PKC-alpha/beta pseudosubstrate peptide, staurosporine Ro-31-7549, Ro-31-8220, Ro-31-8425, Ro-32-0432, sangivamycin; calphostin C, safingol, D-erythro-sphingosine, chelerythrine chloride, melittin; dequalinium chloride, Go6976, Go6983, Go7874, polymyxin B sulfate; cardiotoxin, ellagic acid, HBDDE, 1-O-Hexadecyl-2-O-methyl-rac-glycerol, hypercin, K-252, NGIC-J, phloretin, piceatannol, tamoxifen citrate, flavopiridol, and bryostatin 1.

5. The method of claim 1, wherein the mammal is human.

6. The method of claim 1, wherein the mammal is suffering from the RSV infection, and wherein said decreasing alleviates at least one of the symptoms associated with the RSV infection.

7. The method of claim 1, wherein the mammal is not suffering from the RSV infection, and said decreasing is carried out as prophylaxis against RSV infection.

8. The method of claim 3, wherein the at least one PKC inhibitor is administered to the mammal orally or intranasally.

9. The method of claim 3, wherein the at least one PKC inhibitor is administered with a pharmaceutically acceptable carrier.

10. The method of claim 3, wherein the at least one PKC inhibitor is co-administered with at least one additional anti-viral agent.

11. The method of claim 3, wherein the at least one PKC inhibitor is a chemical compound.

12. The method of claim 3, wherein the at least one PKC inhibitor is a small, apolar, molecule.

13. The method of claim 3, wherein the at least one PKC inhibitor is a pseudosubstrate peptide.

14. The method of claim 3, wherein the at least one PKC inhibitor is a function-blocking antibody, or a fragment thereof.

15. The method of claim 3, wherein the PKC activity is that of PKC alpha.
